(19) United States
(12) Patent Application Publication
(10) Pub. No.: US 2010/0063004 A1
(43) Pub. Date: Mar. 11, 2010

(54) TOPICAL PHARMACEUTICAL COMPOSITION

(76) Inventors: Ranjan Ray Chaudhuri, Parsippany, NJ (US); Satish Ramchandra Dipali, Parsippany, NJ (US); Phillip E. West, Parsippany, NJ (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/516,879

(22) PCT Filed: Dec. 3, 2007
(86) PCT No.: PCT/US07/86234

§ 371 Date: Nov. 9, 2009

Related U.S. Application Data
(60) Provisional application No. 60/868,507, filed on Dec. 4, 2006.

(51) Int. Cl. A61K 31/045; A61K 31/52; A61K 31/675; A61K 31/662; A61K 31/522; A61P 31/22; A61P 25/00

(52) U.S. Cl. 514/86; 514/724; 514/263.38; 514/263.4; 514/120; 514/263.31

(57) ABSTRACT

This invention relates to a novel topical pharmaceutical composition. In particular, this invention is an overnight topical composition for treating cold sores.

### FIELD OF THE INVENTION

This invention relates to a novel topical pharmaceutical composition. In particular, this invention is a topical composition for treating cold sores.

### BACKGROUND OF THE INVENTION

Herpes simplex virus (HSV-1 and HSV-2), commonly referred to as “herpes virus” or “herpes” is an infectious disease which has reached crisis proportions nationally with estimated numbers of infected people at 70%-80% of U.S. population as reported by the American Social Health Association (ASHA) and growing annually by 500,000 people or more. There are two common types of herpes: herpes simplex virus 1 (HSV-1) and herpes simplex virus 2 (HSV-2).

The HSV-1 virus typically causes cold sores. Primary HSV-1 infections frequently occur during infancy or childhood. Infection results from close contact with infected people. The virus can be transmitted by close personal contact, sharing personal products or by utensils. The resultant sores most commonly affect the lips, mouth, nose, chin or cheeks and occur shortly after exposure. The HSV-2 virus typically causes genital sores. Most people get HSV-2 infections following sexual contact with an infected person. Various estimates indicate that the HSV-2 virus affects between 10 and 40 million people in the United States (up to 25% of all sexually active adults in the United States) and up to 400 million people world wide.

Herpes may enter the human body through minuscule breaks in the epidermal tissue usually by contact with an infected host and is marked by eruption of one or more vesicles, usually in groups, following an incubation period of approximately two to ten days. With either type of herpes simplex virus, a new lesion can be spread by merely touching an unaffected part of the body after touching a herpes lesion.

Outbreaks of the herpes virus generally follow a staged progression. The stages are easily identifiable and include prodrome, erythema/papule, blister/vesicles, ulceration, crust and healing. Some of the stages can last less than 24 hours. Prodrome is generally a short period of tingling, itching, numbness or burning with no visible sign of an outbreak. Erythema/papule is characterized by a raised reddened area. Vesicles are the formation of one or more fluid-filled blisters, often in a cluster and usually surrounded by sore, red skin. The ulceration stage is when the blisters open to form painful ulcers or open sores. At the edge of the sore, a soft or hard yellow crust begins to appear. Ulcers and painful, sore, red skin persist through this stage. At the crust stage, weeping sores or ulcers become completely covered by a crust or scab. No open ulcers or blisters are present at this stage. The healing process is manifested by disappearance of the crust or scab, swelling, pain and itching. Skin eruptions due to viral infection, especially herpes viruses, generally have a normal infective course that lasts from 10 to 60 days depending on the exact causative species and anatomical location of the infection. The vesicles can appear anywhere on epithelial tissues including the skin or mucosa, typically appearing on the lips as cold sores, on glands, oral mucosa, conjunctiva and cornea and on genitalia, anal mucosa and peri-anal tissue as herpes genitalis.

Herpes symptoms include: inguinal swelling, pain, fever, malaise, headaches, muscle aches and swollen glands. Oral herpes may impact the trigeminal nerve, causing in some individuals excruciating facial pain, difficulty swallowing and eating and facial swelling. The sores themselves are often painful and unsightly.

Herpes viral infections are chronic. Once the virus enters the body it lies dormant in the nerve cells and periodically reactivates. While the cause of a recurrent herpetic infection is unknown, various triggers are thought to contribute. These triggers include:

exposure to sunlight; nutritional deficiencies; stress; menstruation; immunosuppression; certain foods; drugs; febrile illness; etc. When the virus reactivates, it characteristically causes a sore at the site where it first entered the body. To date, other than for Herpes Varicella-Zoster (chicken pox), there is neither a vaccine to prevent the herpes infection, nor any way to eliminate the virus from the body. Once infected, the patient has the virus for life.

At the present time oral anti-viral medications such as acyclovir, famcyclovir, or valacyclovir, and topical anti-viral medications such as acyclovir and pencyclovir, are available to treat herpes infections. These medications can be used to treat an outbreak or can be used for suppressing herpes recurrences. Lower doses may be helpful in reducing the number of herpes attacks in people with frequent outbreaks.

Prevention of the disease, which is contagious before and during an outbreak, is important. It is known that kissing someone while having a fever blister or having sex with one having an outbreak of genital herpes likely leads to transmission of the virus. However, herpes can be transferred in the absence of lesions or prodromal symptoms (asymptomatic viral shedding).

Most antiviral therapeutic compounds block various specific viral genetic replicative mechanisms within infected target cells. These approaches have drawbacks including toxicity to host cells, induction of drug-resistant viral substrains, and potential to act as mutagens and/or teratogens for host cells. Consequently, the search for new antiviral compounds that provide efficacious therapy, without such deleterious consequences to the host, is of paramount importance.

As early as 1974, n-docosanol, also referred to as 1-docosanol and behenyl alcohol, was reported to have systemic therapeutic value. For example, Debar, U.S. Pat. No. 4,186,211, reported that 1-docosanol when taken orally was therapeutically effective in the treatment of enlargement of the prostate gland. Further studies disclosed in U.S. Pat. No. 4,874,794, teach the use of n-docosanol as a topical antiviral agent. In addition, U.S. Pat. No. 5,534,554, teaches stable and efficacious topical creams containing sucrose cocoate, sucrose stearates or sucrose distearate, or mixtures, thereof, with C-20 to C-28 aliphatic alcohols, e.g. n-docosanol, as the therapeutic active.

n-Docosanol, is a straight chain 22-carbon saturated alcohol, which occurs naturally and is reported to have broad activity in cell cultures against lipid enveloped viruses such as herpes. n-Docosanol is metabolized by the cell membrane and incorporated into the cell wall where it inhibits the ability of enveloped viruses to fuse the viral envelop with the cell membrane. This fusion is critical for the viral DNA to enter the cell. When the viral DNA is kept out, it can not use cellular mechanisms to replicate. Once the virus enters a cell, n-docosanol is not expected to have an effect on viral replication. Pope L E, et al., Antiviral Research 1998:40:85-94.

n-Docosanol is a crystalline waxy solid insoluble in water which to date has typically required composition with a non-ionic surfactant and a carrier to facilitate dermal penetration and interaction at the target cell level (see, for example, U.S. Pat. No. 5,534,554, teaching a topical composition of n-docosanol having a sugar-based ester surfactant in a mineral oil carrier). In a study using 10% n-docosanol suspended in an aqueous system containing a non-ionic surfactant and a carrier, mean healing time of lesions in humans infected with herpes labialis (HSV-1) was shortened (Habbema et al., Acta Derm. Venereol., 76: 479-481, 1996) (Sacks S L, et al., Clinical Efficacy of Topical Docosanol 10% Cream for Herpes Simplex Labialis: A Multicenter, Randomized, Placebo-controlled Trial. J Am Acad Dermatol 2001; 45:222-230).

While significant medical research in this field of endeavor has been focused on developing compositions used for treating tissue affected by HSV, compositions providing rapid relief to these ailments are still needed. It would therefore be an improvement in the art to provide compositions, systems and methods of treating tissue disorders such as those associated with HSV, in particular, cold sores related to infection with HSV-1, that overcome the problems of the prior art.

### SUMMARY OF THE INVENTION

In one aspect, this invention relates to a therapeutic composition for application to the skin and membranes comprising 0.1-25 percent by weight of a C20-C28 aliphatic alcohol, in particular, n-docosanol, a plasticizer, a film-forming polymer and a sugar-based ester surfactant.

In another aspect, this invention relates to a therapeutic composition for application to the skin and membranes comprising 0.1-25 percent by weight of a C20-C28 aliphatic alcohol, in particular, n-docosanol, a plasticizer, a film-forming polymer and a sugar-based ester surfactant, which composition forms a film over the affected area maintaining the therapeutic active in place until removed.

In yet another aspect, this invention relates to a method of treatment of recurrent herpetic episodes in humans, comprising applying a therapeutic amount of a composition for application to the skin and membranes comprising 0.1-25 percent by weight of a C20-C28 aliphatic alcohol, in particular, n-docosanol, a plasticizer, a film-forming polymer and a sugar-based ester surfactant to a human in need thereof.

In still another aspect, this invention relates to a method for reducing the pain of surface inflammation of the skin or membrane, comprising applying to an inflamed surface of the skin or membrane, a therapeutic amount of a composition comprising 0.1-25 percent by weight of a C20-C28 aliphatic alcohol, in particular, n-docosanol, a plasticizer, a film-forming polymer and a sugar-based ester surfactant.

In a further aspect, this invention relates to a method of treatment of HSV-1 in mammals, comprising applying a therapeutic amount of a composition for application to the skin and membranes comprising 0.1-25 percent by weight of a C20-C28 aliphatic alcohol, in particular, n-docosanol, a plasticizer, a film-forming polymer and a sugar-based ester surfactant to a mammal in need thereof and allowing the formation of a protective film over the affected area, or an area believed to be affected by HSV-1.

In yet a further aspect, this invention relates to various regimens for treating, reducing the healing time and reducing the pain associated with recurrent herpetic episodes using the therapeutic composition of this invention.

### DETAILED DESCRIPTION OF THE INVENTION

The present invention provides a topical composition and method of treatment of lesions associated with herpes viral infections of the skin, including HSV-1. A herpes viral outbreak generally progresses through the following stages: prodrome, erythema/papule, vesicles, ulceration, crust and healing. The lesion is commonly referred to as a cold sore.

The term “lesion” as used herein at all occurrences shall refer to any of the stages of a viral outbreak.

The terms “remove” or “removed” as used herein at all occurrences shall refer to the intentional washing off or wiping off of the inventive composition.

The terms “treating” or “treatment” as used herein at all occurrences shall refer to therapeutic therapy.

Application of the instant compositions reduces the healing time of HSV-1 lesions as well as stopping the normal progression of the HSV-1 outbreak from the stage at which the initial application occurred. The present compositions generally reduce the healing time of an HSV-1 outbreak from approximately 10 days to about 4 to 5 days. In one embodiment of the invention, the healing time of an HSV-1 outbreak is 4 days.

The compositions of the present invention are suitable for topical applications and include, without limitation, creams, lotions, gels, ointments or pastes, and the like. The present compositions comprise 0.1-25 percent by weight of a C20-C28 aliphatic alcohol, in particular, n-docosanol, a plasticizer, a film-forming polymer and a sugar-based ester surfactant.

The careful selection of each of these components has provided an optimum anti-viral effect that has also produced some unexpectedly enhanced results. In addition to having a therapeutic effect, the instant compositions are advantageous in that they are particularly suited for night time use of the composition. The compositions dry within 5 to 15 minutes of application. In one embodiment of the invention, the composition dries within 10 minutes of application. In addition, the compositions allow the therapeutic active ingredient to be in contact with the lesion for prolonged times periods until the composition is removed. Unlike other topical compositions, it does not rub off while sleeping and does not need to be reapplied during the treatment time period. In one embodiment, the composition remains on the site of application during the normal course of a night's sleep and certainly for up to eight hours. In a further embodiment, the composition is applied overnight for between about 6 and 10 hours. In another embodiment, the composition remains on the site of application any time of day provided the site remains substantially undisturbed. The inventive compositions are capable of remaining on the site of application for one or more days if undisturbed.

The ability of the body to heal dermal wounds is a highly evolved process. When a wound is created, the body stimulates and precisely regulates a host of activities that are dependent upon the environment surrounding, or within, the wound. In an ideal environment the healing process occurs optimally; however, when a dermal wound is untreated, or poorly treated, the environment is not ideal and dermal healing is compromised. Many factors effect the wound healing environment including protection from the external environment and the amount of moisture and the level of nutrients available to the tissue.

It is believed that an occlusive film provides an ideal environment for the body to activate and maintain the healing process that prevents wound desiccation; this in turn allows easier cell migration, allows growth factors and matrix materials to be more readily available and probably maintains the temperature and electrical gradient of the wound. Miller M C, et al., Biodrugs 2005: 19(6), 363-381. Martin P, et al., Inflammatory cells during wound repair: the good, the bad and the ugly. Trends Cell Biol. 2005:15(11), 599-607. The inventive composition contains film formers that occlude or shield the wound, having beneficial wound healing properties. In addition, occlusive films accelerate epithelialization, alleviate pain and prevent infection. The film forms an occlusive barrier over the lesion providing symptomatic relief of pain, burning, itching, and tingling.

Without being bound to any particular theory, the film-forming properties of the composition are believed to enhance the therapeutic effect of the active ingredient since it is in contact with the infected site until removed. Therefore, the active has a more significant opportunity to act at the lesion site. The composition is easily removed once the user desires to wash it off. A treatment regimen for recurrent herpetic episodes would include applying the composition to the site of infection at the first sign of lesion formation, typically described as a tingling sensation, and then over the course of at least 4 days, and up to 10 days, depending upon the speed to healing of the particular outbreak. The composition is applied to the site of infection before sleep, allowed to dry and then removed upon awakening.

The active therapeutic agents useful in these compositions are C20-C28 aliphatic alcohols. In one embodiment, n-docosanol is the active agent. The amount of the inventive composition required for therapeutic effect on topical administration will, of course, vary with the nature and severity of the condition being treated and is ultimately at the discretion of the physician. A suitable therapeutic dose of an active ingredient according to this invention is wherein the active agent is present in the composition in an amount between 0.1 to 25 percent by weight of the total composition. In one embodiment, the active agent is 10 percent by weight of the total composition. Suitably, the carrier for the composition is mineral oil.

The film-forming polymers suitable for this invention include, but are not limited to polyvinylpyrrolidone (available from ISP, New Jersey), hydroxypropylmethyl cellulose, hydroxypropyl cellulose, and polyvinylacetate. The film-forming polymer is present in an amount which is between 2 and 10 percent by weight of the total composition. In one embodiment, the film-forming polymer is present in an amount which is 5.0 percent by weight of the total composition. In one embodiment of the invention, the film-forming polymer is polyvinylpyrrolidone which is present in an amount which is between 3.0 and 6.0 percent by weight of the total composition. In another embodiment of the invention, the film-forming polymer is polyvinylpyrrolidone which is present in an amount which is between 2.0 and 5.0 percent by weight of the total composition.

The plasticizer present in the composition, includes, but is not limited to glycerin (also known as glycerol). Suitably, the plasticizer is present in an amount which is 0.5 to 5 percent by weight of the total composition. In one embodiment the plasticizer is present in an amount which is 0.9 percent by weight of the total composition.

Suitably, an emulsifier is included and is selected from esterified sugar-based surfactants as described in U.S. Pat. No. 5,534,554, incorporated herein by reference. In one embodiment of this invention, the esterified sugar-based surfactant is sucrose stearate/distearate (available from Croda under the trade name of Crodesta F110). In one embodiment the emulsifier is present in an amount which is 5.0 percent by weight of the total composition.

In addition, one or more excipients suitably may be added to the instant composition. Such excipients include, but are not limited to, a preservative, an opacifying agent, a second therapeutic agent, and a humectant.

Suitably, a preservative may be present in the composition, which preservative includes, but is not limited to benzyl alcohol, benzoic acid, ethylenediaminetetraacetic acid (EDTA), sodium benzoate, methylparaben, propylparaben, butylparaben and sorbic acid. Suitably, the preservative is present in an amount which is 0.05 to 5 percent by weight of the total composition. In one embodiment the preservative is benzyl alcohol and is present in an amount which is 2.7 percent by weight of the total composition.

Suitably, an opacifying agent may be present in the instant composition. The purpose of this agent is to provide visual confirmation that the film remains in place on the site of application. Suitable opacifying agents include, but are not limited to titanium dioxide, zinc oxide, stearene/acrylates and talc. Suitably, the opacifying agent is present in an amount which is 0.2 to 5 percent by weight of the total composition. In one embodiment, the opacifying agent is titanium dioxide and is present in an amount which is 1 percent by weight of the total composition.

Suitably, a further therapeutic agent may be included in the composition. Useful therapeutic agents for these compositions include, but are not limited to anti-viral agents such as acyclovir, famcyclovir, pencyclovir, cidofovir, foscarnet, ganciclovir, valacyclovir, and valgancyclovir. Suitably, the further therapeutic agent is present in the composition in an amount which is 1 to 5 percent by weight of the total composition. It is recognized that in certain forms of therapy, combinations of these agents in the same delivery system may be useful in order to obtain an optimal therapeutic effect.

Suitably, a humectant may be present in the composition, which humectant includes, but is not limited to, propylene glycol, sorbitol, and glycerol triacetate (Triacetin). Suitably, the humectant is present in an amount which is 3 to 15 percent by weight of the total composition. In one embodiment, the humectant is propylene glycol.

### Examples

### Example 1

Compositions included within the scope of this invention are found in the tables below.

TABLE 1

| Item | Ingredient Name | % w/w |
|---|---|---|
| 1 | Light Mineral Oil | 8.00 |
| 2 | n-Docosanol | 10.00 |
| 3 | Purified Water | 65.10 |
| 4 | Sucrose stearate/distearate | 5.00 |
| 5 | Propylene Glycol | 5.00 |
| 6 | Benzyl Alcohol | 2.70 |
| 7 | Glycerol | 0.90 |
| 8 | Titanium Dioxide | 1.00 |
| 9 | Polyvinylpyrrolidone | 2.30 |
| | TOTAL | 100.00% |

TABLE 2

| Item | Ingredient Name | % w/w |
|---|---|---|
| 1 | Light Mineral Oil | 8.00 |
| 2 | n-Docosanol | 10.00 |
| 3 | Purified Water | 62.40 |
| 4 | Sucrose stearate/distearate | 5.00 |
| 5 | Propylene Glycol | 5.00 |
| 6 | Benzyl Alcohol | 2.70 |
| 7 | Glycerol | 0.90 |
| 8 | Titanium Dioxide | 1.00 |
| 9 | Polyvinylpyrrolidone | 5.00 |
| | TOTAL | 100.00% |

TABLE 3

| Item | Ingredient Name | % w/w |
|---|---|---|
| 1 | Light Mineral Oil | 8.00 |
| 2 | n-Docosanol | 10.00 |
| 3 | Purified Water | 59.30 |
| 4 | Sucrose stearate/distearate | 5.00 |
| 5 | Propylene Glycol | 8.00 |
| 6 | Benzyl Alcohol | 2.70 |
| 7 | Glycerol | 1.00 |
| 8 | Titanium Dioxide | 1.00 |
| 9 | Polyvinylpyrrolidone | 5.00 |
| | TOTAL | 100.00% |

TABLE 4

| Item | Ingredient Name | % w/w |
|---|---|---|
| 1 | Light Mineral Oil | 8.00 |
| 2 | n-Docosanol | 10.00 |
| 3 | Purified Water | 64.30 |
| 4 | Sucrose stearate/distearate | 5.00 |
| 5 | Propylene Glycol | 5.00 |
| 6 | Benzyl Alcohol | 2.70 |
| 7 | Glycerol | 1.00 |
| 8 | Titanium Dioxide | 1.00 |
| 9 | Polyvinylpyrrolidone | 3.00 |
| | TOTAL | 100.00% |

TABLE 5

| Item | Ingredient Name | % w/w |
|---|---|---|
| 1 | Light Mineral Oil | 8.00 |
| 2 | n-Docosanol | 10.00 |
| 3 | Acyclovir | 5.00 |
| 4 | Purified Water | 57.40 |
| 5 | Sucrose stearate/distearate | 5.00 |
| 6 | Propylene Glycol | 5.00 |
| 7 | Benzyl Alcohol | 2.70 |
| 8 | Glycerol | 0.90 |
| 9 | Titanium Dioxide | 1.00 |
| 10 | Polyvinylpyrrolidone | 5.00 |
| | TOTAL | 100.00% |

TABLE 6

| Item | Ingredient Name | % w/w |
|---|---|---|
| 1 | Light Mineral Oil | 8.00 |
| 2 | n-Docosanol | 10.00 |
| 3 | Acyclovir | 5.00 |
| 4 | Purified Water | 58.30 |
| 5 | Sucrose stearate/distearate | 5.00 |
| 6 | Propylene Glycol | 5.00 |
| 7 | Benzyl Alcohol | 2.70 |
| 8 | Glycerol | 1.00 |
| 9 | Titanium Dioxide | 1.00 |
| 10 | Polyvinylpyrrolidone | 4.00 |
| | TOTAL | 100.00% |

TABLE 7

| Item | Ingredient Name | % w/w |
|---|---|---|
| 1 | Light Mineral Oil | 8.00 |
| 2 | n-Docosanol | 10.00 |
| 3 | Acyclovir | 5.00 |
| 4 | Purified Water | 54.30 |
| 5 | Sucrose stearate/distearate | 5.00 |
| 6 | Propylene Glycol | 8.00 |
| 7 | Benzyl Alcohol | 2.70 |
| 8 | Glycerol | 1.00 |
| 9 | Titanium Dioxide | 1.00 |
| 10 | Polyvinylpyrrolidone | 5.00 |
| | TOTAL | 100.00% |

TABLE 8

| Item | Ingredient Name | % w/w |
|---|---|---|
| 1 | Light Mineral Oil | 8.00 |
| 2 | n-Docosanol | 10.00 |
| 3 | Pencyclovir | 5.00 |
| 4 | Purified Water | 57.40 |
| 5 | Sucrose stearate/distearate | 5.00 |
| 6 | Propylene Glycol | 5.00 |
| 7 | Benzyl Alcohol | 2.70 |
| 8 | Glycerol | 0.90 |
| 9 | Titanium Dioxide | 1.00 |
| 10 | Polyvinylpyrrolidone | 5.00 |
| | TOTAL | 100.00% |

### Example 2

### Preparation of a Composition with the Components Found in Table 9:

TABLE 9

| Item | Ingredient Name | % w/w |
|---|---|---|
| 1 | Light Mineral Oil | 8.00 |
| 2 | n-Docosanol | 10.00 |
| 3 | Purified Water | 61.80 |
| 4 | Sucrose stearate/distearate | 5.00 |
| 5 | Propylene Glycol | 5.00 |
| 6 | Benzyl Alcohol | 2.70 |
| 7 | Glycerol | 1.00 |
| 8 | Titanium Dioxide | 1.50 |
| 9 | Polyvinylpyrrolidone | 5.00 |
| | TOTAL | 100.00% |

### Aqueous Phase:

Water (55.62% w/w; 299.7 mg) was added to a vessel, and using an impeller mixer with a wide blade (adjusted to the vessel size) was dispersed the sucrose stearate/distearate (5% w/w; 20.00 mg) at about 30° C. This dispersion was stirred slowly (10% speed) and heated to a temperature between 75-85° C. While the temperature of the dispersion was increasing, and when the dispersion reaches a temperature between 55-70° C., propylene glycol (5.00% w/w; 30.1 mg) was added. The scraper blade was set to a speed of 10. These contents were homogenized at 20% homogenizer speed for 2 minutes.

Benzyl alcohol (2.70% w/w; 16.2 mg) was added to the above mixture while the temperature was between 75-85° C. At this stage, the speed of the scraper blade was increased to 30% for 1 minute. The contents were seen to begin clearing, with an apparent reduction in viscosity. The vessel was removed from the heat source and the polyvinylpyrrolidone (5.00% w/w; 30.0 mg), glycerol (1.00% w/w; 6.00 mg) and titanium dioxide (1.50% w/w; 9.1 mg) were added with dispersion using an impeller mixer.

Oil phase:

In a separate vessel, n-docosanol (10.00% w/w; 60.1 mg) and mineral oil (8.00% w/w; 48.0 mg) were combined and heated to a temperature between 75-85° C. Once these contents were at the required temperature, the oil phase was added to the aqueous phase while washing the oil phase vessel with the remaining water (6.18% w/w; 33.3 mg) at the same temperature.

### Homogenization:

During the addition of the oil phase to the aqueous phase, the scraper blade was set to 30% and the homogenizer to 20 speeds, for 3 minutes. After this, the vessel was allowed to cool to 25° C. while allowing the homogenization to continue for an additional 3 minutes. At this point, the homogenizer was removed and the scraper blade continued to mix at 10% speed. The contents were allowed to fully cool to 25° C. for approximately an additional 150 minutes.

### Example 3

A composition according to this invention can be manufactured according to the following process.

Transfer oil ingredients of the formula into a suitable vessel and heat ingredients to between 75-85° C. to the required temperature while mixing at suitable speed. Once melted, hold this melted mixture aside for further use. In a separate suitable vessel, transfer the purified water and turn on agitator at an appropriate speed and heat the water to between 35-85° C. Once the water is at the required temperature, turn the homogenizer to an appropriate speed and pull a vacuum. Transfer the emulsifier into this batch. After transfer, release the vacuum, and mix the batch until the emulsifier is fully dispersed. Transfer the plasticizer (and preservative and/or humectant, if used), under vacuum. Allow sufficient time for the ingredients to mix with this batch. Transfer the film-forming polymer and opacifying agent under vacuum. Release vacuum and continue mixing the aqueous batch at appropriate agitator and homogenizer speed until they are mixed homogeneously into the batch. While mixing, increase the temperature of the batch to final processing temperature between 75-85° C.

Ensure that the temperatures of the oil and aqueous batches in the vessels are within 5° C. of one another before blending. Transfer the batch with oil ingredients into the aqueous batch. If required pull vacuum. Mix the combination batch at an appropriate homogenizer and agitator speed until the product is homogeneous. Slowly begin cooling the combination batch to room temperature. Discharge the product into an appropriate holding vessel and hold it in storage area for further use.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

What is claimed is:

1. A therapeutic composition for topical application to skin and membranes comprising 0.1-25 percent by weight of a C20-C28 aliphatic alcohol, a plasticizer, a film-forming polymer and a sugar-based ester surfactant.

2. The therapeutic composition as claimed in claim 1, wherein the C20-C28 aliphatic alcohol is n-docosanol.

3. The therapeutic composition as claimed in claim 1, further comprising an opacifying agent.

4. The therapeutic composition as claimed in claim 3, wherein the opacifying agent is selected from titanium dioxide, zinc oxide, stearene/acrylates and talc.

5. The therapeutic composition as claimed in claim 4 wherein the opacifying agent is titanium dioxide.

6. The therapeutic composition as claimed in claim 5, wherein titanium dioxide is present in an amount between 0.2 to 5 percent by weight of the total composition.

7. The therapeutic composition as claimed in claim 1, wherein the film-forming polymer is polyvinylpyrrolidone.

8. The therapeutic composition as claimed in claim 7, wherein polyvinylpyrrolidone is present in an amount between 2.0 and 10.0 percent by weight of the total composition.

9. The therapeutic composition as claimed in claim 1, further comprising a second therapeutic agent selected from acyclovir, famcyclovir, pencyclovir, cidofovir, foscarnet, ganciclovir, valacyclovir, and valgancyclovir.

10. The therapeutic composition as claimed in claim 1, wherein the plasticizer is glycerine.

11. A method of treating recurrent herpetic episodes in humans comprising topically administering a therapeutic amount of a composition comprising 0.1-25 percent by weight of a C20-C28 aliphatic alcohol, a plasticizer, a film-forming polymer and a sugar-based ester surfactant to a human in need thereof.

12. The method as claimed in claim 11, wherein the C20-C28 aliphatic alcohol is n-docosanol.

13. A method of treating symptoms associated with a cold sore lesion comprising topically administering a therapeutic amount of a composition comprising 0.1-25 percent by weight of a C20-C28 aliphatic alcohol, a plasticizer, a film-forming polymer and a sugar-based ester surfactant to a person in need thereof.

14. The method as claimed in claim 13, wherein the C20-C28 aliphatic alcohol is n-docosanol.

15. A therapeutic composition for topical application to a herpes virus lesion comprising 0.1-25 percent by weight of a C20-C28 aliphatic alcohol, a plasticizer, a film-forming polymer and a sugar-based ester surfactant, which composition forms a film on the lesion maintaining the composition on the lesion until removed.

16. The method as claimed in claim 15, wherein the C20-C28 aliphatic alcohol is n-docosanol.

18. A method for reducing pain associated with surface inflammation of the skin or membrane, comprising topically administering to an inflamed surface of the skin or membrane, a therapeutic amount of a composition comprising 0.1-25 percent by weight of a C20-C28 aliphatic alcohol, a plasticizer, a film-forming polymer and a sugar-based ester surfactant.

19. The method as claimed in claim 18, wherein the C20-C28 aliphatic alcohol is n-docosanol.

20. A method of protecting a cold sore lesion from physical disturbance during healing, comprising topically administering to the lesion a therapeutic amount of a composition comprising 0.1-25 percent by weight of n-docosanol, a plasticizer, a film-forming polymer and a sugar-based ester surfactant.

21. The method as claimed in claim 20, wherein the C20-C28 aliphatic alcohol is n-docosanol.

22. The method of claim 21 further comprising applying the therapeutic composition overnight for between about 6 and 10 hours.

23. A regimen for treating recurrent herpetic episodes in humans comprising topically administering a therapeutic amount of a composition comprising 0.1-25 percent by weight of a C20-C28 aliphatic alcohol, a plasticizer, a film-forming polymer and a sugar-based ester surfactant to a human in need thereof; allowing the composition to remain undisturbed at the site of application for between 6 and 10 hours; removing the composition; and repeating the procedure over the course of at least 4 days.

24. The regimen as claimed in claim 23, wherein the C20-C28 aliphatic alcohol is n-docosanol.

25. A regimen for reducing the healing time of a lesion associated with recurrent herpetic episodes in humans, comprising topically administering to the lesion a therapeutic amount of a composition comprising 0.1-25 percent by weight of a C20-C28 aliphatic alcohol, a plasticizer, a film-forming polymer and a sugar-based ester surfactant to a human in need thereof; allowing the composition to remain undisturbed at the site of application for between 6 and 10 hours; removing the composition; and repeating the procedure over the course of at least 4 days.

26. The regimen as claimed in claim 25, wherein the C20-C28 aliphatic alcohol is n-docosanol.

27. A regimen for reducing pain associated with surface inflammation of the skin or membrane, comprising topically administering to an inflamed surface of the skin or membrane, a therapeutic amount of a composition comprising 0.1-25 percent by weight of a C20-C28 aliphatic alcohol, a plasticizer, a film-forming polymer and a sugar-based ester surfactant to a human in need thereof; allowing the composition to remain undisturbed for between 6 and 10 hours; removing the composition; and repeating the procedure over the course of at least 4 days.

28. The regimen as claimed in claim 27, wherein the C20-C28 aliphatic alcohol is n-docosanol.

29. A therapeutic composition for topical application to skin and membranes comprising 10.0 percent by weight of a C20-C28 aliphatic alcohol, 1.0 percent by weight of a plasticizer, 5.0 percent by weight of a film-forming polymer and 5.0 percent by weight of a sugar-based ester surfactant.

30. A therapeutic composition for topical application to skin and membranes comprising 10.0 percent by weight of n-docosanol, 1.0 percent by weight of a plasticizer, 5.0 percent by weight of a film-forming polymer and 5.0 percent by weight of a sugar-based ester surfactant.

31. A therapeutic composition for topical application to skin and membranes consisting essentially of 8.00% w/w of mineral oil, 10.00% w/w of n-docosanol, 65.10% w/w water, 5.00% w/w of sucrose stearate/distearate, 5.00% w/w of propylene glycol, 2.70% w/w of benzyl alcohol, 0.90% w/w of glycerol, 1.00% w/w of titanium dioxide, and 2.30% w/w of polyvinylpyrrolidone.
